(19) United States
(12) Patent Application Publication
(10) Pub. No.: US 2014/0288416 A1
(43) Pub. Date: Sep. 25, 2014

(54) MANIPULATION OF AN UNTETHERED MAGENTIC DEVICE WITH A MAGNET ACTUATOR

(71) Applicant: University of Utah Research Foundation, Salt Lake City, UT (US)

(72) Inventors: Arthur W. Mahoney, Salt Lake City, UT (US); Jacob J. Abbott, Salt Lake City, UT (US)

(21) Appl. No.: 14/223,510

(22) Filed: Mar. 24, 2014

Related U.S. Application Data
(60) Provisional application No. 61/852,855, filed on Mar. 22, 2013.
(60) Provisional application No. 61/837,055, filed on Jun. 19, 2013.

(51) Int. Cl. A61B 1/00; A61B 1/04

(52) U.S. Cl. CPC A61B 1/00158; A61B 1/041
USPC 600/424; 600/118

(57) ABSTRACT

A method of manipulating an untethered magnetic device, such as a magnetic capsule endoscope, can include positioning a magnet actuator and an untethered magnetic device proximate one another such that the untethered magnetic device is within a constant magnetic field of the magnet actuator. A position of the untethered magnetic device can be determined relative to the magnet actuator. A desired heading, position, and/or velocity of the untethered magnetic device can be identified. The method can include calculating a magnetic field heading and/or a magnetic force to be applied to the untethered magnetic device to achieve the desired device heading, position, and/or velocity. The magnetic actuator can be moved to apply the calculated magnetic field heading and/or magnetic force to the untethered magnetic device via the magnetic field. The magnetic field heading can be operable to orient the untethered magnetic device in the desired heading and the magnetic force can be operable to translate the untethered magnetic device to the desired position and/or at the desired velocity such that the magnetic device can levitate above a surface.

### RELATED APPLICATIONS

This application claims the benefit of U.S. Provisional Application No. 61/852,855, filed Mar. 22, 2013, and U.S. Provisional Application No. 61/837,055, filed Jun. 19, 2013, each of which is incorporated herein by reference.

### GOVERNMENT INTEREST

This invention was made with government support under Grant #0952718 awarded by the National Science Foundation. The Government has certain rights in the invention.

### BACKGROUND

Magnetic manipulation of capsule endoscopes has the potential to make current gastrointestinal screening procedures faster, safer, and less invasive. To date, three electromagnetic systems have been developed with the ability to perform five degree-of-freedom (5-DOF) manipulation of an untethered magnetic device, such as a magnetic capsule endoscope. The MAGNETECS and OCTOMAG systems consist of eight electromagnets arranged around a sphere and hemisphere, respectively, and directed toward the manipulation workspace. A system has been developed by Siemens, consisting of 12 electromagnets through which a patient is positioned, for the control of a capsule endoscope in a water-filled stomach. Permanent-magnet actuation systems are gaining attention for their ability to generate fields with clinically relevant strengths, inexpensively and in a compact form-factor, compared to electromagnetic systems.

Previous permanent-magnet systems for endoscopy have been limited to dragging and rolling capsule endoscope devices on the stomach's surface or large colon. For example, such permanent magnet systems cannot levitate a capsule in space, such as when moving the capsule, while also controlling the capsule's heading. Heading and, typically, translation have previously been done when the capsule was on a surface when utilizing permanent magnets or constant magnetic fields. Accordingly, improvements continue to be sought to avoid such drawbacks. The present technology provides magnetic 3-DOF position and 2-DOF orientation control of a stomach capsule endoscope in fluid, using a magnet providing a constant magnetic field. Manipulation of untethered devices with 5-DOF has been previously demonstrated with electromagnet systems with variable magnetic fields.

### SUMMARY

In one aspect, the present technology provides a method of manipulating a magnetic capsule endoscope or other untethered magnetic device in 3-D space when not in contact with a surface. The method can include positioning a magnet actuator and an untethered magnetic device proximate one another such that the untethered magnetic device is within a constant magnetic field of the magnet actuator. The method can also include determining a position of the untethered magnetic device relative to the magnet actuator. The method can further include identifying a desired heading, position, and/or velocity of the untethered magnetic device. Still further, the method can include calculating a magnetic field heading and/or a magnetic force to be applied to the untethered magnetic device to achieve the desired heading, position, and/or velocity. In addition, the method can include moving the magnetic actuator to apply the calculated magnetic field heading and/or magnetic force to the untethered magnetic device via the magnetic field. The magnetic field heading can be operable to orient the untethered magnetic device to the desired heading (like a compass needle) and the magnetic force can be operable to translate the untethered magnetic device to the desired position and/or at the desired velocity as the magnetic device levitates above a surface.

The present technology can also provide a computer implemented method of controlling a magnet actuator to manipulate an untethered magnetic device. The method can be performed under control of a processor and memory configured with executable instructions. The method can include identifying a relative position of an untethered magnetic device and a magnet actuator, the untethered magnetic device being within a constant magnetic field of the magnet actuator. The method can also include identifying a desired heading, position, and/or velocity of the untethered magnetic device. The method can further include calculating a magnetic field heading and/or a magnetic force to be applied to the untethered magnetic device to achieve the desired heading, position, and/or velocity. In addition, the method can include transmitting instructions to move the magnet actuator to apply the calculated magnetic field heading and/or magnetic force to the untethered magnetic device via the magnetic field. The magnetic field heading can be operable to orient the untethered magnetic device to the desired heading and the magnetic force can be operable to translate the untethered magnetic device to the desired position and/or at the desired velocity as the untethered magnetic device levitates above a surface.

In another aspect, the present technology provides a system for manipulating an untethered magnetic device. The system can include a magnet actuator configured to generate a constant magnetic field to influence a heading, a position, and/or a velocity of an untethered magnetic device. The system can also include a localization device to determine a position of the untethered magnetic device relative to the magnet actuator. The system can further include a control system to calculate a magnetic field heading and/or a magnetic force to be applied to the untethered magnetic device to achieve a desired device heading, position, and/or velocity of the untethered magnetic device. In addition, the system can include a movement device configured to move the magnetic actuator to apply the calculated magnetic field heading and/or magnetic force to the untethered magnetic device via the magnetic field. The magnetic field heading can be operable to orient the untethered magnetic device in the desired heading and the magnetic force can be operable to translate the untethered magnetic device to the desired position and/or at the desired velocity as the magnetic device levitates above a surface.

Additional variations and aspects of the invention can be appreciated from the following detailed description.

### BRIEF DESCRIPTION OF THE DRAWINGS

FIG. 1 is an illustration of a system for manipulating an untethered magnetic device, in accordance with an example of the present disclosure.

FIG. 2 is an illustration of a magnet actuator of the system of FIG. 1, in accordance with an example of the present disclosure.

FIG. 3 is an illustration of an untethered magnetic device of the system of FIG. 1, in accordance with an example of the present disclosure.

FIGS. 4A and 4B are illustrations of actuator magnet poses along with the resulting total force vectors applied to an untethered magnetic device.

FIG. 4C illustrates resulting manipulator poses for select total force vectors of FIGS. 4A and 4B.

FIGS. 5A-5C show an untethered magnetic device's heading rotated from a down-pointing to a side-pointing configuration, while maintaining a stationary position in space.

FIGS. 6A-6C show an untethered magnetic device's position following a U-shaped trajectory while maintaining constant heading.

FIGS. 7A-7C show an untethered magnetic device moved from right to left along a square-wave path while maintaining a constant heading.

FIG. 8 shows a desired square-wave trajectory and the actual paths traveled by the untethered magnetic device of FIGS. 7A-7C while moving at different desired spatial velocities.

FIGS. 9A-9C show an untethered magnetic device performing a remote center-of-motion (RCM) maneuver, requiring the untethered magnetic device's position to rotate around (while simultaneously pointing at) a fixed point in space.

FIG. 10A shows 3D position-tracking performance of the untethered magnetic device of FIGS. 9A-9C for different localization rates.

FIG. 10B shows heading-tracking performance of the untethered magnetic device of FIGS. 9A-9C.

These figures are provided merely for convenience in describing specific embodiments of the invention. Alteration in dimension, materials, and the like, including substitution, elimination, or addition of components can also be made consistent with the following description and associated claims. Reference will now be made to the exemplary embodiments illustrated, and specific language will be used herein to describe the same. It will nevertheless be understood that no limitation of the scope of the invention is thereby intended.

### DETAILED DESCRIPTION

Reference will now be made to certain examples, and specific language will be used herein to describe the same. Examples discussed herein set forth a system for manipulating an untethered magnetic device and associated methods that can orient and translate an untethered magnetic device with a constant magnetic field as the magnetic device levitates above a surface.

With the general embodiments set forth above, it is noted that when describing a system for manipulating an untethered magnetic device, or the related methods, each of these descriptions are considered applicable to the other, whether or not they are explicitly discussed in the context of that embodiment. For example, in discussing the system per se, the method embodiments are also included in such discussions, and vice versa.

It is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to “a processor” includes one or more of such processors.

Also, it is noted that various modifications and combinations can be derived from the present disclosure and illustrations, and as such, the following figures should not be considered limiting.

In describing and claiming the present invention, the following terminology will be used in accordance with the definitions set forth below.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, “heading” denotes a pointing orientation of the device without regard to the device's rotation around the pointing axis.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation can apply regardless of the breadth of the range or the characteristics being described.

Any steps recited in any method or process claims may be executed in any order and are not limited to the order presented in the claims unless otherwise stated. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

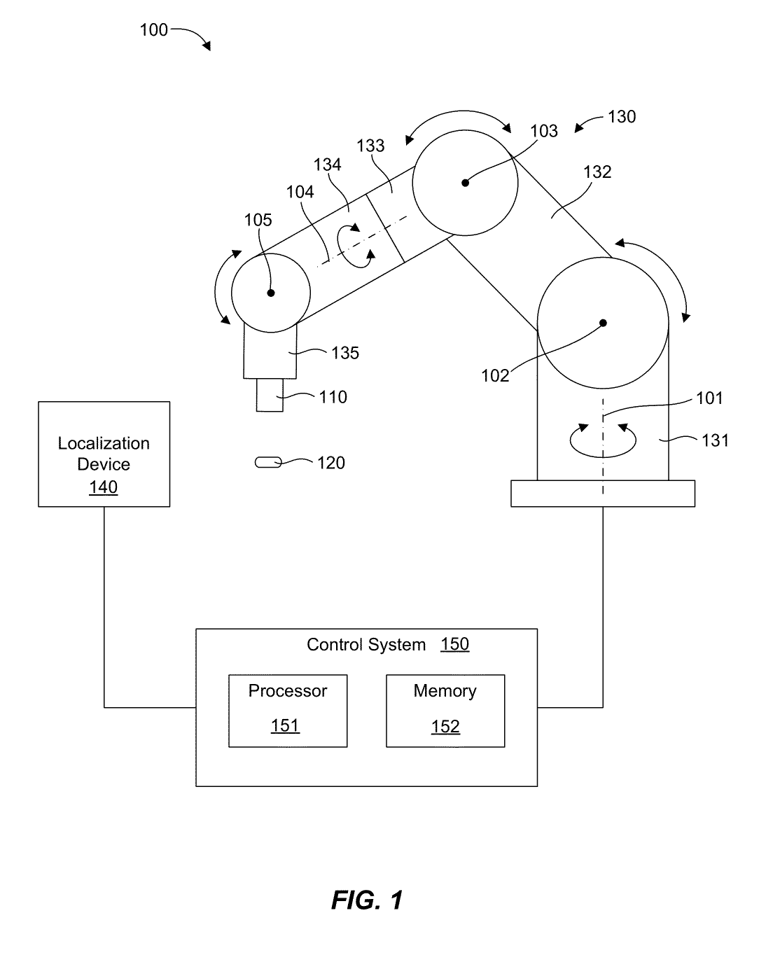

One example of a system 100 for manipulating an untethered magnetic device 120 is illustrated in FIG. 1. The system can include a magnet actuator 110 and an untethered magnetic device 120 that is controllable by the magnet actuator, as described herein, when the magnet actuator and the magnetic device are positioned proximate one another such that the untethered magnetic device is within a magnetic field of the magnet actuator. In one aspect, the magnet actuator can be configured to generate a constant magnetic field to influence heading, a position, and/or a velocity of the untethered magnetic device.

The magnet actuator 110 can be moved and manipulated by movement device 130, such as a robotic manipulator. Thus, in one aspect, the magnet actuator can be considered an end effector for the robotic manipulator. The robotic manipulator can be configured to move the magnet actuator in multiple degrees of freedom (DOF). For example, as shown in the figure, the robotic manipulator can be configured to move in 5-DOF. In particular, the robotic manipulator illustrated is configured to provide rotational degrees of freedom for various segments or linkage arms of the robotic manipulator. In this case, the robotic manipulator is configured to rotate a first linkage arm 131 about a first axis 101, a second linkage arm 132 about a second axis 102, a third linkage arm 133 about an axis 103, a fourth linkage arm 134 about an axis 104, and a fifth linkage arm 135 about an axis 105. The magnet actuator can be coupled to the fifth linkage arm, thus being movable in the 5-DOF provided by the robotic manipulator to control the untethered magnetic device, as disclosed in more detail hereinafter.

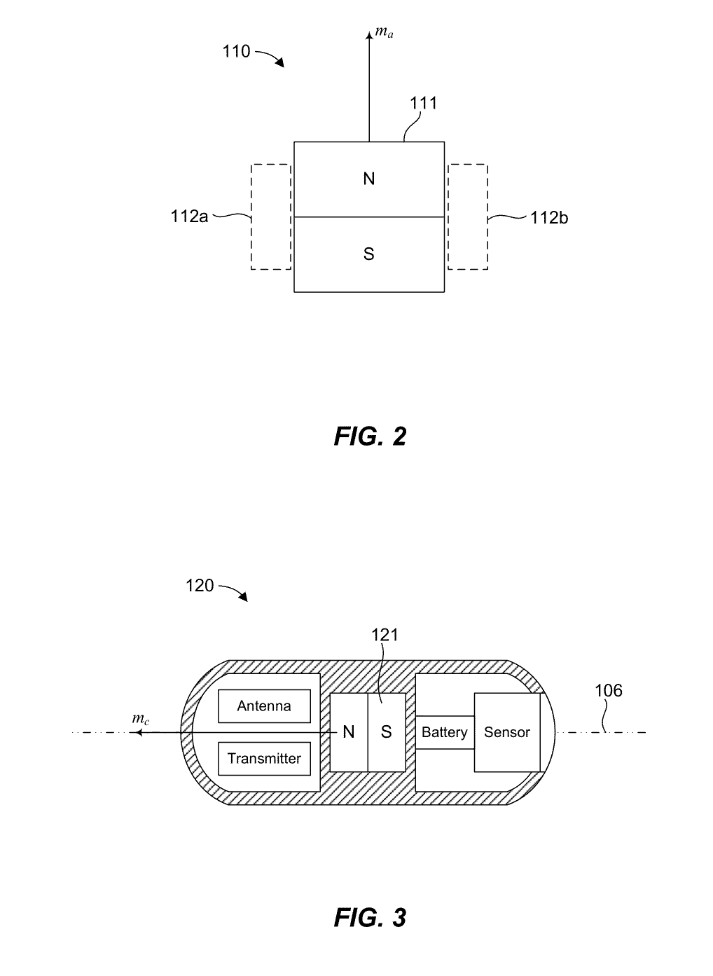

With continued reference to FIG. 1, an example of the magnet actuator 110 is shown in FIG. 2. The magnet actuator can comprise any suitable permanent magnet of any suitable configuration, such as a cylindrical NdFeB magnet. However, other magnets such as, but not limited to, samarium-cobalt and ferrite can also be used. Other magnet geometries can include, but are not limited to, spherical, rectangular, and cubic magnets.

As illustrated in the figure, the magnet actuator can comprise an axially magnetized magnet 111, which has a dipole moment mₐ. The magnet actuator can include only a single permanent magnet or multiple permanent magnets having a composite magnetic dipole. In one aspect, the magnet actuator can comprise an electromagnet having a constant magnetic field when energized or “on,” as opposed to a varying magnetic field when energized, which is often the case with electromagnets.

In one optional aspect, the magnet actuator 110 can include one or more secondary electromagnets 112a, 112b, in addition to the primary magnet 111. In this case, the primary magnet can provide “coarse” control of the untethered magnetic device 120 and the secondary electromagnets can provide “fine” corrections to the coarse control of the permanent magnet. Thus, the secondary electromagnets can provide a relatively small magnetic force to cause a small amount of fine correction to the heading, position, and/or velocity of the untethered magnetic device, instead of having to move the robotic arm to achieve the same result. In another aspect, the secondary electromagnets can be positioned nearby the actuator magnet on a stationary platform near the untethered device sufficient to affect movement of the untethered device.

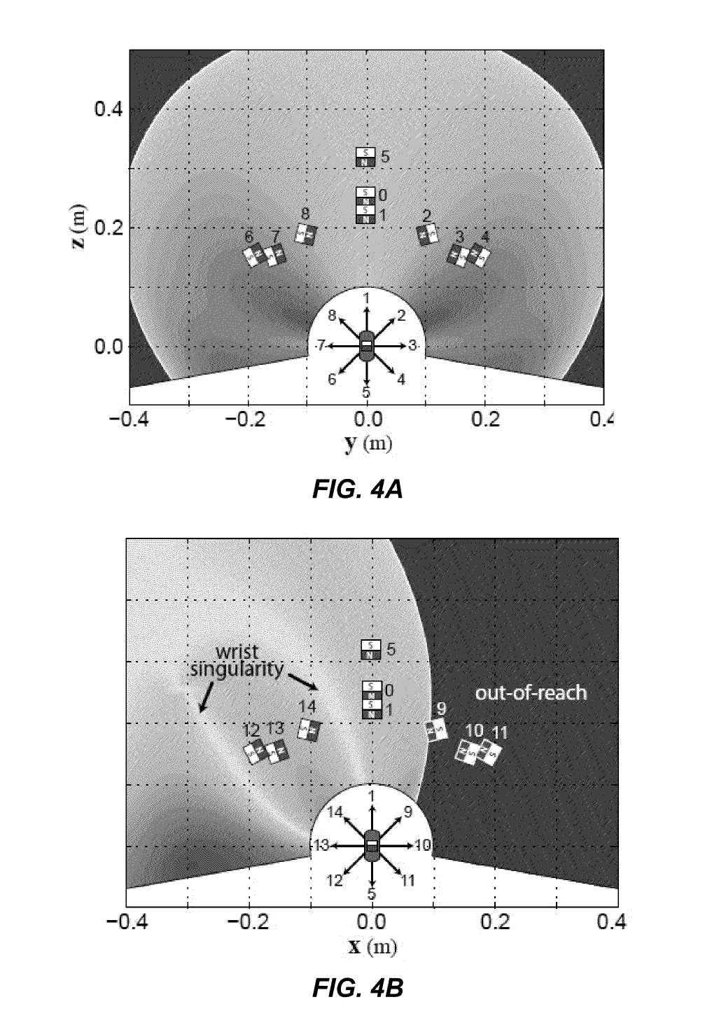

With continued reference to FIGS. 1 and 2, an example of the untethered magnetic device 120 is shown in FIG. 3. The untethered magnetic device can comprise any suitable permanent magnet 121 and can have any suitable configuration, such as a capsule configuration. The magnet can comprise any suitable permanent magnet of any suitable configuration, such as a cube NdFeB magnet, although other permanent magnet materials and geometries can also be used as with the actuator. The magnet can include only a single permanent magnet or multiple permanent magnets. As shown in FIG. 3, the magnet 121 can comprise an axially magnetized magnet, which has a dipole moment m_{c}. The dipole moment can be oriented in any suitable manner with respect to the capsule. In this case, the dipole moment m_{c} of the magnet is arranged parallel to a longitudinal axis 106 of the capsule. Thus, the permanent magnet can have opposing poles positioned toward opposing ends of the capsule. In one aspect, the permanent magnet can have a dipole moment arranged perpendicular to the longitudinal axis. Thus, the permanent magnet can have opposing poles positioned across a diameter of the capsule. In one aspect, the untethered magnetic device 120 can be configured as a magnetic capsule endoscope. For example, the untethered magnetic device can include a sensor, such as a camera/imager, an RF transmitter, an antenna, a battery, or any other suitable feature or device that may be included in a magnetic capsule endoscope. Not only can the systems and methods disclosed herein find application in magnetic capsule endoscopy, such as in a fluid-distended stomach, but the systems and methods of the present disclosure can also be applied to magnetic manipulation in general.

The dipole moments mₐ and m_{c} of the magnet actuator and the untethered magnetic device, respectively, are discussed in more detail hereinafter with regard to a method of manipulating the untethered magnetic device.

With reference to FIG. 1, the system 100 can also include a localization device 140 to determine a position of the untethered magnetic device 120 relative to the magnet actuator 110. The localization device can also determine an orientation of the untethered magnetic device, although this is not necessary to control the untethered magnetic device, as explained further below. The localization device can utilize RF triangulation, magnetics, optics, CT scan, x-ray fluoroscopy, ultrasound, or combinations thereof, or any other suitable device or method to determine an orientation of the untethered magnetic device, as the methods disclosed herein are not dependent on any specific localization modality.

In addition, the system 100 can include a control system 150 for controlling the untethered magnetic device 120 with the magnet actuator 110. The control system can include a processor 151 and memory 152 to calculate a magnetic field heading and/or a magnetic force to be applied to the untethered magnetic device to achieve a desired heading, position, and/or velocity of the untethered magnetic device. The desired heading, position, and/or velocity of the untethered magnetic device can be provided by a human user and/or by the processing system.

In use, the localization device 140 can determine a position of the untethered magnetic device 120, which can be used to identify the relative position of the magnet actuator 110 and the untethered magnetic device. A user can then input, such as via a keyboard, mouse, joystick, controller, etc., a desired heading, position, and/or velocity of the untethered magnetic device. The control system can then calculate a magnetic field heading and/or a magnetic force to be applied to the untethered magnetic device to achieve a desired heading, position, and/or velocity of the untethered magnetic device. With this information, the movement device 130, such as the robotic manipulator, can move the magnetic actuator, as appropriate, to apply the calculated magnetic field heading and/or magnetic force to the untethered magnetic device via the magnetic field. In contrast to other manipulation systems utilizing a constant magnetic field, the magnetic field heading can be operable to orient the untethered magnetic device in the desired heading and the magnetic force can be operable to translate the untethered magnetic device to the desired position and/or at the desired velocity as the magnetic device levitates above a surface. In other words, the untethered magnetic device can be caused to change heading and move while levitating or being suspended against gravity without solid physical contact. As described in more detail below, this kind of control of the untethered magnetic device can be achieved by decoupling calculations of the magnetic field heading and the magnetic force. Thus, although inherently coupled in a physical sense, the magnetic field heading, which provides the desired device heading, can be modeled and calculated independent of the magnetic force, which provides the desired position and/or velocity of the untethered magnetic device.

In one aspect of the present disclosure, a method of manipulating the untethered magnetic device is disclosed, which can be utilized by the system 100 disclosed hereinabove. The untethered magnetic device, referred to as a capsule hereinafter, is assumed to contain a magnet with a constant magnetic field, such as a permanent magnet, positioned at the capsule's center-of-gravity, with its dipole moment (i.e., the vector from the south to north pole) denoted by m_{c}ε [symbol] ³ in units A·m², which is assumed to be parallel to the capsule's principle axis. The actuator magnet's dipole moment is denoted by mₐε [symbol] ³ and is positioned by a robotic manipulator with at least 5-DOF; rotation of the actuator magnet about mₐ is not needed. The positions of the actuator and capsule endoscope magnet centers are denoted by pₐε [symbol] ³ and p_{c}ε [symbol] ³, respectively, in units m.

It is assumed that the magnetic field h(p, \hat{m}ₐ)ε [symbol] ³ generated by the actuator magnet can be modeled by the point-dipole model, which is given by (1)

$$h ( p , {\hat{m}}_{a} ) = \frac{‖ {m}_{a} ‖}{4 π {‖ p ‖}^{3}} D ( p ) {\hat{m}}_{a} , \qquad ( 1 )$$

where p=p_{c}−pₐ is the vector from the center of the actuator magnet to the center of the capsule's magnet (i.e., the relative position), D(\hat{p})=3\hat{p}\hat{p}^{T}−I, and Iε [symbol] ^{3×3} is the identity matrix. Since the magnitudes of mₐ and m_{c} are constant (they are the dipole moments of permanent magnets), all functions of mₐ and m_{c} are expressed as functions of \hat{m}ₐ and \hat{m}_{c} to explicitly indicate that their magnitudes do not vary. Equation (1) exactly predicts the field produced by a spherical magnet and is an approximation for every other geometry that becomes more accurate with increasing distance. The geometry of a nonspherical magnet can be adjusted to make equation (1) a more accurate approximation in the near-field. It is assumed that the dipole field accurately models the field of the actuator magnet.

The robot manipulator's n revolute and prismatic joint velocities \dot{q}ε [symbol] ⁿ are mapped to the actuator magnet's spatial \dot{p}ₐ and angular ωₐ velocities by the robot manipulator Jacobian matrix J_{R}(q)ε [symbol] ^{6×n} as given by (2):

$$[ \begin{matrix}{\dot{p}}_{a} \\ {ω}_{a}\end{matrix} ] = {J}_{ℛ} ( q ) \dot{q} . \qquad ( 2 )$$

The point-dipole field equation (1) is radially symmetric about the actuator dipole moment and any component of ωₐ in the direction of \hat{m}ₐ produces no change in the magnetic field applied to the capsule. As a result, the robot manipulator Jacobian J_{R}(q) can be converted into an actuator-magnet Jacobian matrix J_{A}(q) that maps manipulator joint velocity \dot{q} to the actuator magnet's spatial velocity \dot{p}ₐ and the actuator dipole moment's directional velocity {dot over (\hat{m}ₐ=ωₐ×\hat{m}ₐ, with no contribution from the component of ωₐ parallel to \hat{m}ₐ, by (3)

$$[ \begin{matrix}{\dot{p}}_{a} \\ {{m}^{\dot{\hat{}}}}_{a}\end{matrix} ] = [ \begin{matrix}I & 0 \\ 0 & {S ( {\hat{m}}_{a} )}^{T}\end{matrix} ] {J}_{ℛ} ( q ) \dot{q} = {J}_{A} ( q ) \dot{q} , \qquad ( 3 )$$

where S(\hat{m}ₐ)εso(3) is the skew-symmetric form of the cross-product operation. The matrix J_{A}(q) can be used to approximately map small changes in the manipulator's joints to small changes in actuator-magnet position and small changes in the heading of the actuator magnet's dipole moment, as given by (4):

$$[ \begin{matrix}δ {p}_{a} \\ δ {\hat{m}}_{a}\end{matrix} ] \approx {J}_{𝒜} ( q ) δ q . \qquad ( 4 )$$

Note that J_{A}(q) is not invertible and is at most rank five.

When the capsule is placed in the magnetic dipole field (1) generated by the actuator magnet, a magnetic torque Tₘ=μ₀m_{c}×h(p, \hat{m}ₐ) and force fₘ=μ₀(m_{c}·Δ)h(p, \hat{m}ₐ) are applied to the capsule's magnet, which are given by (5) and (6), respectively,

$${τ}_{m} ( p , {\hat{m}}_{a} , {\hat{m}}_{c} ) = \frac{{μ}_{0} ‖ {m}_{a} ‖ ‖ {m}_{c} ‖}{4 π {‖ p ‖}^{3}} {\hat{m}}_{c} \times D ( \hat{p} ) {\hat{m}}_{a} \\ {f}_{m} ( p , {\hat{m}}_{a} , {\hat{m}}_{c} ) = \frac{3 {μ}_{0} ‖ {m}_{a} ‖ ‖ {m}_{c} ‖}{4 π {‖ p ‖}^{4}} ( {\hat{m}}_{a} {\hat{m}}_{c}^{T} + {\hat{m}}_{c} {\hat{m}}_{a}^{T} + ( {\hat{m}}_{c}^{T} Z {\hat{m}}_{a} ) I ) \hat{p} , \qquad ( 5 , 6 )$$

where Z=I−5\hat{p}\hat{p}^{T} and μ₀=4π×10⁻⁷\,N·A⁻² is the permeability of free-space. The magnetic torque aligns the capsule's dipole moment with the applied field, while the magnetic force pulls the capsule in a direction determined by the field's spatial derivatives and the capsule's dipole moment.

When the magnetic capsule is actuated in fluid at low speeds, small accelerations, and without contact with other objects, there is little resistance to change in the capsule's heading, which enables the magnetic torque to quickly align the capsule's dipole moment with the applied field. In these conditions, it is assumed that the capsule's dipole moment is approximately aligned with the applied field for all time (7):

\hat{m}_{c}(p,\hat{m}ₐ)≈ĥ(p,\hat{m}ₐ)= [symbol] (7)

and the capsule's heading can be controlled by adjusting the direction of the magnetic field without controlling the magnetic torque directly using (5), which would require measurement of the direction of \hat{m}_{c} (i.e., the capsule's heading). It also implies that \hat{m}_{c} can be predicted by (7) using only a measurement of the position p obtained by a localization system, and that the magnetic force applied to the magnetic capsule can be predicted by substituting (7) into (6) to get (8):

$${f}_{m} ( p , {\hat{m}}_{a} ) \approx \frac{3 {μ}_{0} ‖ {m}_{a} ‖ ‖ {m}_{c} ‖}{4 π {‖ p ‖}^{3} ‖ D ( \hat{p} ) {\hat{m}}_{a} ‖} ( {\hat{m}}_{a} {\hat{m}}_{a}^{T} - ( 1 + 4 {( {\hat{m}}_{a}^{T} \hat{p} )}^{2} ) I ) \hat{p} . \qquad ( 8 )$$

The total force f applied to the capsule consists of the apparent weight f_{w} (sum of the capsule's weight and buoyant force), which is constant, and the magnetic force fₘ. It is assumed that the capsule is heavier than its buoyant force, making f_{w} point in the direction of gravity. In this case, the capsule can be made to levitate by positioning the actuator magnet above the capsule, where the attractive magnetic force perfectly balances the capsule's apparent weight and the magnitude of the total applied force f is zero. If the capsule is desired to ascend, then the actuator magnet is moved closer so that the magnetic force is larger than the capsule's apparent weight and the total applied force is directed upward. If the capsule is desired to descend, then the actuator magnet is positioned farther away from the capsule's levitation position and the total applied force points down. The maximum downward force that can be applied is the capsule's apparent weight f_{w}.

Using (7) and (8), a nonlinear magnetic actuation equation (9) can be formed that relates the relative position p and the direction of the actuator magnet \hat{m}ₐ to the total applied force f and the direction of the applied magnetic field ĥ:

$$[ \begin{matrix}f \\ \hat{h}\end{matrix} ] = [ \begin{matrix}{f}_{m} ( p , {\hat{m}}_{a} ) + {f}_{w} \\ {a}_{}\end{matrix} ] = ℱ ( p , {\hat{m}}_{a} ) , \qquad ( 9 )$$

which is purely a function of the actuator magnet's pose, that is, the relative position p and the actuator magnet's dipole moment direction \hat{m}ₐ, which in turn, are purely specified by the capsule's position p_{c} and the robot manipulator's pose q.

In order to solve the “inverse” problem (i.e., computing the necessary manipulator pose that will apply a desired total applied force and an applied magnetic field heading, given the capsule's position), the nonlinear actuation equation (9) is first linearized with the Jacobian matrix J_{F}ε [symbol] ^{6×6}, computed by differentiating (9) with respect to the relative position p and the actuator dipole moment \hat{m}ₐ. Linearization produces the approximate mapping between small changes in relative position and actuator moment direction to small changes in the applied force and field heading, as shown in (10) and (11):

$$\begin{matrix}[ \begin{matrix}δ f \\ δ \hat{h}\end{matrix} ] \approx {J}_{ℱ} ( p , {\hat{m}}_{a} ) [ \begin{matrix}δ p \\ δ {\hat{m}}_{a}\end{matrix} ] \\ = {J}_{ℱ} ( p , {\hat{m}}_{a} ) ( [ \begin{matrix}δ {p}_{c} \\ 0\end{matrix} ] + [ \begin{matrix}- I & 0 \\ 0 & I\end{matrix} ] [ \begin{matrix}δ {p}_{a} \\ δ {\hat{m}}_{a}\end{matrix} ] ) ,\end{matrix} \qquad ( 10 , 11 )$$

where (11) results from substituting δp=δp_{c}−δpₐ into (10). The relation (11) divides a small change in applied total force and applied field heading into the result of a small change in capsule position δp_{c} and a small change in the actuator magnet's pose (i.e., the actuator-magnet position δpₐ and dipole heading δ\hat{m}ₐ), which is related to small changes in the manipulator's joints by the Jacobian J_{A} (4). Linearization can be one way to decouple calculations of the magnetic field heading and the magnetic force. Another alternative approach is an iterated explorative method that first translates the actuator magnet using equation 10 to change the applied force, and then using an estimate of the capsule's new position in space, using equation 9 to adjust the actuator magnet heading to achieve the desired capsule heading.

Substituting (4) into (11) produces the relationship between small changes in the manipulator's joints and capsule position to small changes in applied total force and field heading, as shown in (12) and (13):

$$\begin{matrix}[ \begin{matrix}δ f \\ δ \hat{h}\end{matrix} ] \approx {J}_{ℱ𝒜} ( p , q ) δ q + {J}_{ℱ} ( p , {\hat{m}}_{a} ) [ \begin{matrix}δ {p}_{c} \\ 0\end{matrix} ] & ( 12 ) \\ {J}_{ℱ𝒜} ( p , q ) = {J}_{ℱ} ( p , {\hat{m}}_{a} ) [ \begin{matrix}- I & 0 \\ 0 & I\end{matrix} ] {J}_{𝒜} ( q ) . & ( 13 )\end{matrix}$$

The actuator magnet's dipole moment \hat{m}ₐ does not appear in the arguments of J_{FA} since \hat{m}ₐ is set by the robot manipulator's joints q using the manipulator's forward kinematics.

Equation (12) is intended to be used inside a control loop where small changes in capsule position δp_{c} are obtained by a capsule localization system, and δf and δĥ are small desired changes produced by a controller governing the magnetic capsule's pose. In this context, the terms of (12) can be rearranged to produce (14)

$$δ d = [ \begin{matrix}δ f \\ δ \hat{h}\end{matrix} ] - {J}_{ℱ} ( p , {\hat{m}}_{a} ) [ \begin{matrix}δ {p}_{c} \\ 0\end{matrix} ] \approx {J}_{ℱ𝒜} ( p , q ) δ q , \qquad ( 14 )$$

where δd is a desired change in applied force and field heading resulting only from a change in the manipulator's joints. Equation (14) can be inverted to produce the inverse mapping of desired change in applied force and a change in field heading to a necessary change in the manipulator's joints using the Moore-Penrose pseudoinverse, as in (15):

δq≈J_{FA}(p,q)^{\}δd. (15)

If multiple solutions of (15) are possible (i.e., the manipulator has more than 5-DOF), then the pseudoinverse solves (15) and minimizes |δq|. (A generalized pseudoinverse can be applied for a manipulator where the units of δq are inconsistent.) Given an initial joint configuration q₀, (15) can be integrated in time to produce qₜ without explicitly solving the inverse kinematics of the complete manipulator-magnet system. This approach breaks down when the manipulator is near a kinematic singularity, which is address hereinafter.

For 5-DOF holonomic control, J_{FA}(p, q) must be rank five. Since J_{FA}(p, q) is the product of J_{F}(p, \hat{m}ₐ) and J_{A}(q), we will analyze the rank of the Jacobian J_{F}(p, \hat{m}ₐ) and the Jacobian J_{A}(q) separately. For readability, the Jacobians J_{FA}(p, q), J_{F}(p, \hat{m}ₐ) and J_{A}(q) are referred to without their arguments in the text (i.e., as J_{FA}, J_{F}, and J_{A}).

Prior to analyzing the rank of J_{F}, we first scale the columns and rows of J_{F} to produce a nondimensional Jacobian \tilde{J}_{F} that approximately maps its preimage, consisting of nondimensional changes in position δp/|p| and changes in actuator magnet heading δ\hat{m}ₐ (already nondimensional), to its image, consisting of nondimensional changes in force δf/|fₘ| and applied field heading δĥ (already nondimensional), as in (16):

$${\tilde{J}}_{ℱ} ( p , {\hat{m}}_{a} ) = [ \begin{matrix}\frac{1}{‖ {f}_{m} ‖} I & 0 \\ 0 & I\end{matrix} ] {J}_{ℱ} [ \begin{matrix}‖ p ‖ I & 0 \\ 0 & I\end{matrix} ] \qquad ( 16 )$$

where Iε [symbol] ^{3×3} is the identity matrix.

The nondimensional Jacobian \tilde{J}_{F} is produced by post- and premultiplying J_{F} with a series of elementary matrices, which guarantees that rank \tilde{J}_{F}=rank J_{F} and enables the rank of J_{F} to be found using the singular value decomposition of \tilde{J}_{F} with unit-consistent singular values, which reveal the rank of J_{F}. Since the applied field direction ĥ cannot change in a direction parallel to itself, the smallest singular value σ₆ must be zero. The second smallest singular value σ₅ reveals whether the rank of rank \tilde{J}_{F}=5. The minimum value taken on by σ₅ is 0.123, indicating that \tilde{J}_{F} (and thus J_{F}) is always rank five.

The fact that J_{F} is always rank five implies that a single permanent magnet in space, irrespective to the robot manipulator that maneuvers it, can exhibit 5-DOF control over an untethered magnetic device. The ability of a complete robotic system, including magnet and manipulator, to exhibit 5-DOF magnetic control is precluded only by the ability of the robot manipulator to position the actuator magnet with 3-DOF and the actuator magnet's dipole moment with 2-DOF. If the rank of the Jacobian J_{A} is five, then the robotic system possesses 5-DOF control over the untethered capsule. If the actuator-magnet pose required to achieve a desired applied total force and magnetic field heading places the manipulator into a kinematic singularity, then 5-DOF magnetic control is lost.

The configurations of total forces and field headings that make the manipulator enter a singularity are numerically analyzed by first nondimensionalizing the Jacobian J_{A} as (17)

$${\tilde{J}}_{𝒜} ( q ) = [ \begin{matrix}\frac{1}{‖ p ‖} I & 0 \\ 0 & I\end{matrix} ] {J}_{𝒜} ( q ) , \qquad ( 17 )$$

which can then be substituted, along with \tilde{J}_{F}, into (13) for J_{F} and J_{A} to produce the normalized Jacobian (18)

$${\tilde{J}}_{ℱ𝒜} ( p , q ) = {\tilde{J}}_{ℱ} ( p , {\hat{m}}_{a} ) [ \begin{matrix}- I & 0 \\ 0 & I\end{matrix} ] {\tilde{J}}_{𝒜} , ( q ) \qquad ( 18 )$$

which approximately maps change in manipulator joints δq (already nondimensional) to change in nondimensional applied force and change in field heading (already nondimensional).

The Moore-Penrose pseudoinverse \tilde{J}_{FA}^{\} is the inverse mapping that minimizes |δq| if the robot manipulator is over-actuated. The largest singular value of \tilde{J}_{FA}^{\} (i.e., the reciprocal of the smallest nonzero singular value of \tilde{J}_{FA}) can be used to describe the worst case of how a unit-magnitude vector of nondimensional change in applied force and field heading are approximately mapped to a magnitude change in manipulator joints. If the largest singular value approaches infinity, then the robot manipulator is near a kinematic singularity.

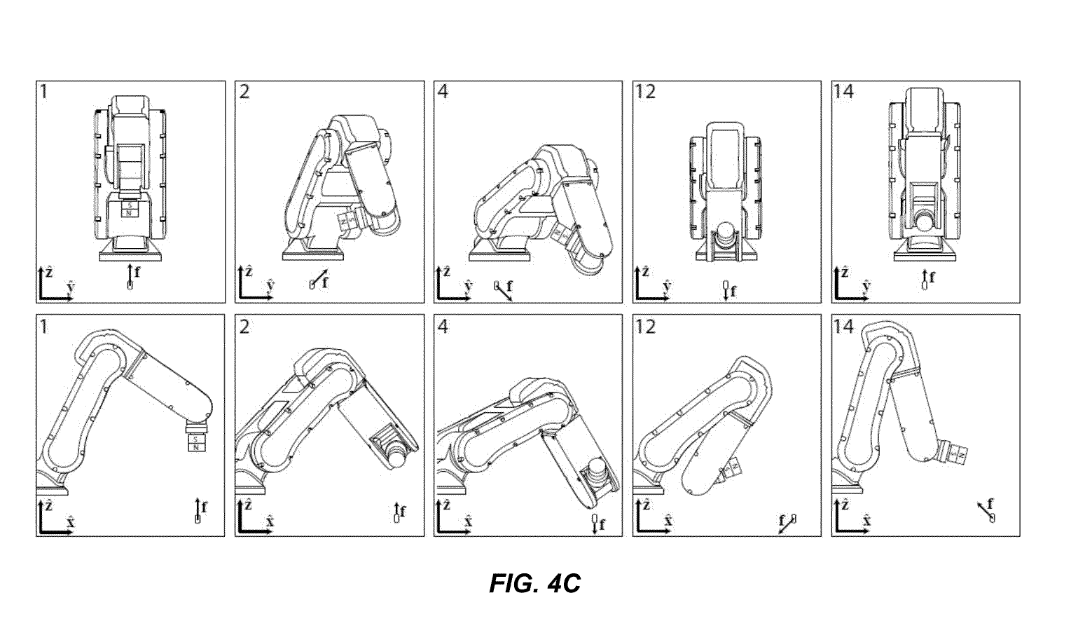

As an example, illustrated in FIGS. 4A-4C, consider the case where a magnetic capsule is desired to point downward (in the direction of gravity) as it is being repositioned by an applied magnetic force, and the robotic manipulator used to maneuver the actuator magnet is a 6-DOF serial link manipulator. In this case, it is assumed that the magnetic capsule, actuator magnet, and robotic manipulator are those used for the example disclosed hereinafter and the capsule is placed in a typical position in front of the manipulator. In FIGS. 4A and 4B, the manipulator's workspace has been sliced into \hat{x},\hat{z} and ŷ,\hat{z} planes. For every possible actuator magnet position on both planes, the actuator magnet's dipole direction \hat{m}ₐ is set to guarantee that the applied field at the capsule position points in the −\hat{z} direction. The necessary heading of the actuator magnet's dipole moment \hat{m}ₐ is given by (19)

\hat{m}ₐ=− [symbol] \hat{z}, (19)

where D⁻¹(\hat{p})=(D(\hat{p})−I)/2 admits a unique actuator magnet pose for every actuator magnet position.

The largest singular value of \tilde{J}_{FA}^{\} resulting from the robot manipulator configuration that places the actuator magnet in every feasible position in the \hat{x},\hat{z} and ŷ,\hat{z} planes and directs the actuator magnet's moment according to (19) are shown in FIGS. 4A and 4B. The regions where the manipulator nears its spherical-wrist kinematic singularity cause the singular value to become large. The regions outside the manipulator's reachable workspace are shown in gray.

Each actuator magnet pose causes a magnetic force to be applied to the capsule. Fifteen numbered actuator magnet poses are illustrated in FIGS. 4A and 4B along with the resulting correspondingly numbered total force vectors applied to the capsule. Each force vector denotes a total force magnitude of |f|=0.3 mN. The resulting manipulator poses for select numbered total force vectors are shown in FIG. 4C. The pose that balances the applied magnetic force and the capsule's apparent weight is labeled as pose “0.” The manipulator's physical workspace limits and kinematic singularities complicate which forces the system can apply. For example, due to workspace limits of the manipulator, forces labeled “9,” “10,” and “11” are not achievable, and due to the manipulator's wrist singularity, transitions from the capsule levitation configuration (pose “0”) to a 0.3 mN force in the −\hat{x} direction (pose “13”) would require the manipulator to pass through its wrist singularity. Note that the direction of the actuator magnet's dipole moment satisfies (19) and applies a magnetic field in the −\hat{z} direction at the capsule's position.

In one aspect, a manipulator's motion near kinematic singularities can be managed, while applying differential kinematic inversion. A strategy can be implemented that sacrifices control over the capsule's heading in order to maintain control over the magnetic force applied to the capsule (thus its position) in the presence of a manipulator singularity. In other words, the ability to control the untethered magnetic device's heading can be sacrificed to maintain control over the untethered magnetic device's position when a robotic manipulator moving the magnetic actuator enters a kinematic singularity. Sacrificing heading control transforms the complete magnetic manipulation system into one that is kinematically over-actuated.

Given a small desired change in applied field heading δĥ_{d} and a small desired change in applied magnetic force δf_{d}, the problem of sacrificing heading control, while maintaining control over the applied magnetic force, is posed as a constrained, quadratic least-squares problem, of the form of (20), (21), and (22), respectively,

$${minimize}_{δ q} ‖ \frac{\partial \hat{h}}{\partial q} δ q - δ {\hat{h}}_{d} ‖ \\ subject to \\ \frac{\partial f}{\partial q} δ q = δ {f}_{d} \\ ‖ W δ q ‖ \leq r , \qquad ( 20 , 21 , 22 )$$

which is solved numerically, where the matrices ∂f/∂qε [symbol] ^{3×n} and ∂ĥ/∂qε [symbol] ^{3×n} are the top and bottom three rows of the Jacobian J_{FA}, respectively. The constraint (21) guarantees the desired change in applied force δf_{d} is met (provided ∂f/∂q has full row rank), and the constraint (22) enforces a maximum bound r on the magnitude of joint motion, weighted by the invertible matrix W. The cost function (20) attempts to reduce the error between the desired and actual change in applied field heading. The weight matrix W can be used to penalize select joint motions, to homogenize disparate units of δq, or to keep the magnitude of δq within a “trust-region,” where the Jacobian J_{FA} is accurate. Note that if the magnitude constraint (22) is inactive (e.g. if the robot manipulator is not near a kinematic singularity) and J_{FA} is rank-five, then the solution to the formulation (20)-(22) is equivalent to the solution obtained with the pseudoinverse (15).

There are two ways for the formulation (20)-(22) to break down. The first is if the matrix ∂f/∂q does not have full row rank and the constraint (21) is not satisfiable. The second is if the constraints (21) and (22) become mutually exclusive, which could occur if ∂f/∂q is ill-conditioned, |δf_{d}| is too large, or r is too small for the required joint motion δq.

In one aspect, the weight matrix can be selected with the robotic manipulator in mind. For example, if the matrix reduces the components that correspond to change in relative position p, then the resulting solution will favor large values of p, requiring large movements of the robot end-effector. In another aspect, the weights can be adjusted based on the observed behavior of the robot manipulator. This weighting scheme can be useful when no change in capsule heading or position is desired. The weight matrix can be constant, or it can change over time (e.g., as a function of the configuration of the robotic manipulator). If the weight matrix is a function of the manipulator's velocity Jacobian matrix that relates the actuator dipole moment and relative position p to changes in the robot joint angles, then the weight matrix could be made to penalize combinations of both the actuator dipole moment and p that produce the most change in joint configuration. Such a strategy could be implemented that forces the controller to reduce the joint motion of the robot.

In accordance with one example of the present disclosure, a method of manipulating an untethered magnetic device is provided. The method can include positioning a magnet actuator and an untethered magnetic device proximate one another such that the untethered magnetic device is within a constant magnetic field of the magnet actuator. The method can also include determining a position of the untethered magnetic device relative to the magnet actuator. The method can further include identifying a desired heading, position, and/or velocity of the untethered magnetic device. Still further, the method can include calculating a magnetic field heading and/or a magnetic force to be applied to the untethered magnetic device to achieve the desired heading, position, and/or velocity. In addition, the method can include moving the magnetic actuator to apply the calculated magnetic field heading and/or magnetic force to the untethered magnetic device via the magnetic field. The magnetic field heading can be operable to orient the untethered magnetic device in the desired heading and the magnetic force can be operable to translate the untethered magnetic device to the desired position and/or at the desired velocity as the magnetic device levitates above a surface.

In another example of the present disclosure, a computer implemented method of controlling a magnet actuator to manipulate an untethered magnetic device is provided. The method can be performed under control of a processor and memory configured with executable instructions. The method can include identifying a relative position of an untethered magnetic device and a magnet actuator, the untethered magnetic device being within a constant magnetic field of the magnet actuator. The method can also include identifying a desired heading, position, and/or velocity of the untethered magnetic device. The method can further include calculating a magnetic field heading and/or a magnetic force to be applied to the untethered magnetic device to achieve the desired device heading, position, and/or velocity. In addition, the method can include transmitting instructions to move the magnet actuator to apply the calculated magnetic field heading and/or magnetic force to the untethered magnetic device via the magnetic field. The magnetic field heading can be operable to orient the untethered magnetic device in the desired heading and the magnetic force can be operable to translate the untethered magnetic device to the desired position and/or at the desired velocity as the magnetic device levitates above a surface. It is noted that no specific order is required in the methods disclosed herein, though generally in one embodiment, the method steps can be carried out sequentially.

The methods disclosed herein involve 3-DOF localization of the capsules position, but does not require an estimate of the capsule's heading. In one aspect, the position of the magnetic capsule endoscope can be estimated by calculating an estimated solution based on an environment model. Typically, even physical measurement of position can be noisy. As such, approximation and estimators can be utilized (e.g., a Kalman filter) that uses knowledge of the devices dynamics to filter out noise in the sensor. The estimator can take in measurements as one of its inputs. The estimator can produce estimates of the capsule's position and optionally orientation in continuous space, or the estimator can act as a finite state machine (e.g., the capsule is either on the stomach floor or the capsule is at the fluid surface), or the estimator can produce finite states in some degrees of freedom and continuous estimates in other degrees of freedom. Thus, state-space estimators and Bayesian methods can be used.

### EXAMPLES

A mockup capsule was actuated in a tank of water by an axially magnetized, grade N42, cylindrical NdFeB magnet with a height of 31.75 mm, a diameter of 31.75 mm, and with a dipole moment of |mₐ|=26.2 A·m², positioned by a Yaskawa-Motoman MH5 6-DOF robotic manipulator. The capsule contained a cube NdFeB permanent magnet with its dipole moment |m_{c}|=0.126 A·m² arranged parallel to the capsule's principal axis. The remainder of the capsule's volume was filled with air. The capsule's weight was 15.3 mN and the buoyancy force in water was 14.8 mN. The position of the capsule was triangulated by two orthogonal Basler A602FC cameras, which were used with an extended Kalman filter for capsule-position feedback. Unless otherwise stated, the localization system's update frequency was 90 Hz. The experimental setup of the robot manipulator, vision system, mockup capsule, and the actuator magnet was similar to that shown in FIG. 1.

A PID feedback controller (using the triangulated capsule position) with a gravity-compensating feed-forward term, was implemented to servo the capsule to any desired position in the workspace. At every iteration, the PID controller took as input a desired capsule position p_{c,d}, an estimated capsule position p̅_{c}, and an estimated capsule spatial velocity {dot over ( p̅_{c}, and produced a desired change in applied force δf, which was then combined with a desired change in applied field heading δĥ_{d} and converted into robot manipulator motion by solving the constrained least-squares formulation (20)-(22) with an identity weight matrix W=Iε [symbol] ^{6×6} unless otherwise noted. An estimate of the capsule's heading was obtained from the measured position using (1), and was controlled in an open-loop fashion. A desired change in field heading (which is equivalent a desired change in capsule heading) was computed as the difference between a desired capsule heading and an estimate of the current capsule heading, which was assumed to be small at each controller iteration. Due to a limitation of the commercial manipulator control system, the robot manipulator's position was updated at 25 Hz.

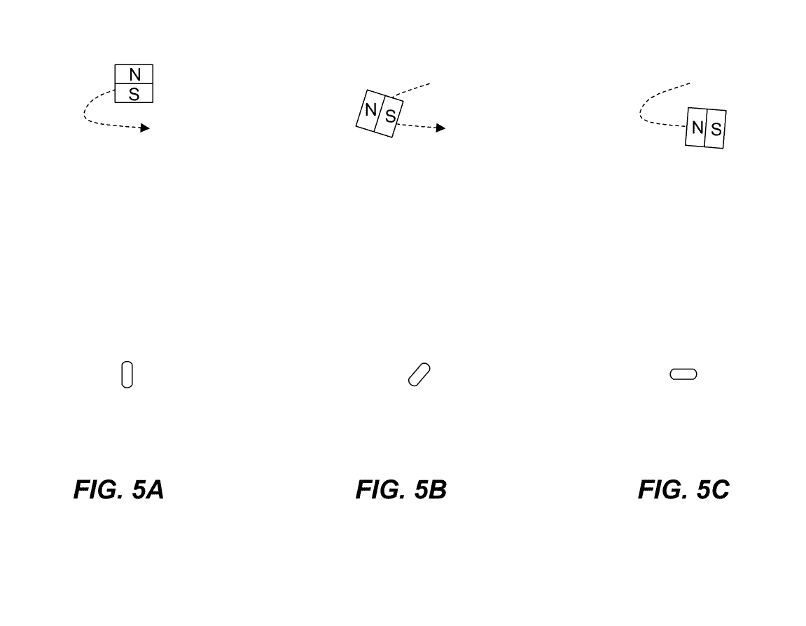

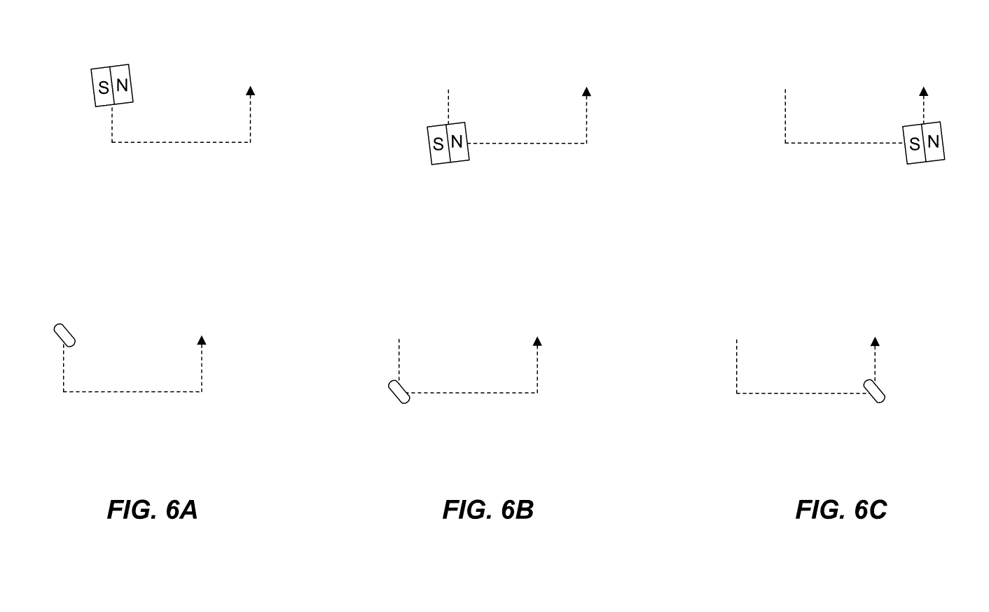

The theory presented herein, as well as the control system, was demonstrated by controlling the magnetic mockup capsule along multiple predefined trajectories. FIGS. 5A-6C show two capsule maneuvers achieved by the present technology that prior actuation systems using a single permanent magnet are unable to perform. For example, in FIGS. 5A-5C, the capsule's heading rotated from a down-pointing to a side-pointing configuration, while maintaining a stationary position in space (i.e., levitating) with no external contact. Nonintuitively, the actuator magnet did not remain directly above the capsule during the transition. In FIGS. 6A-6C, the capsule's position following a U-shaped trajectory while maintaining constant heading.

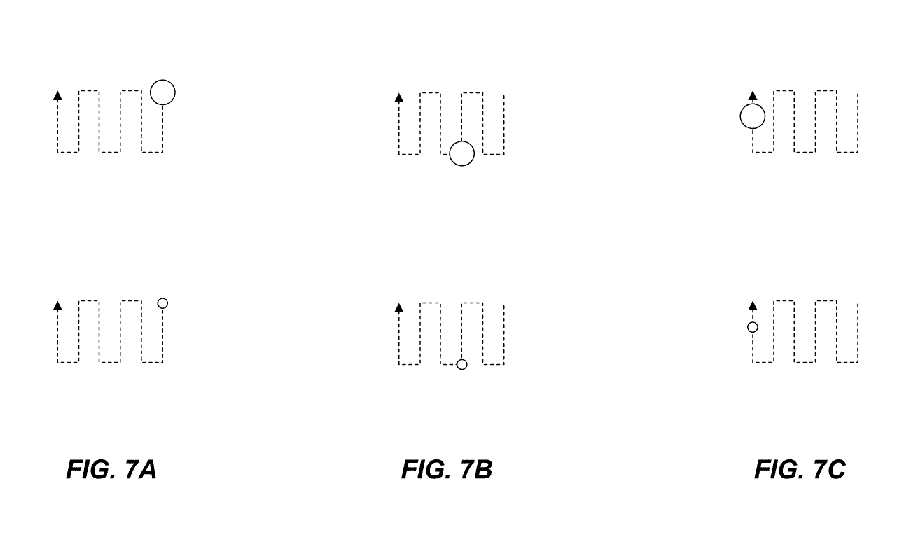

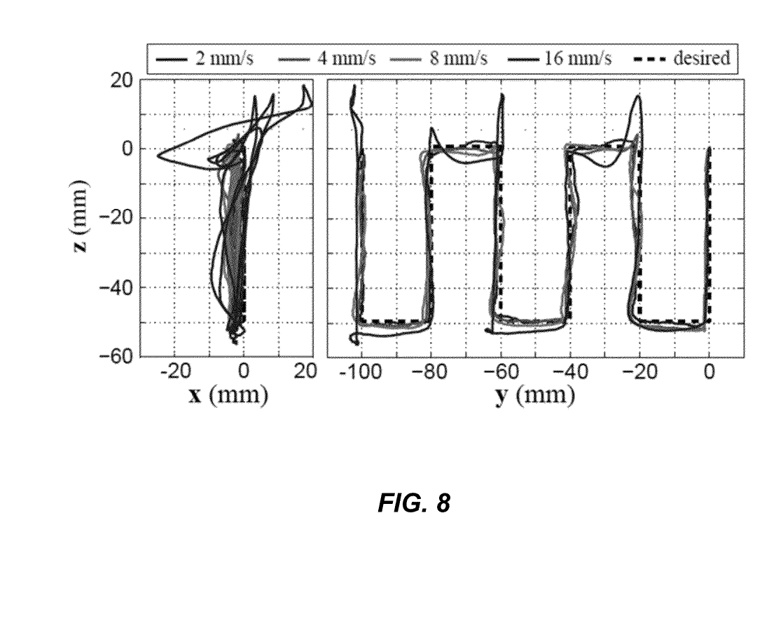

FIGS. 7A-7C show an image sequence of the capsule following a raster-scan trajectory where the capsule moved from right to left along a square-wave path with an amplitude of 50 mm and a period of 40 mm. Longitudinal axes of the magnet actuator and the capsule extend out of the page. Such a trajectory could be used to perform automated visual coverage of a surface for inspection tasks. The desired square-wave trajectory and the actual paths traveled by the capsule while moving at desired spatial velocities of 2 mm/s, 4 mm/s, 8 mm/s, and 16 mm/s are shown in FIG. 8. In general, the trajectory tracking performance was good at slow speeds and worsened with increasing desired spatial velocity. It is important to note that at high spatial velocities, a fluidic torque can be generated that may cause the capsule's dipole moment m_{c} to become misaligned with the applied magnetic field h. If this occurs, then the assumptions discussed above are violated and the actual applied magnetic force may deviate from expected. This occurred to the capsule when following the square-wave trajectory with a desired spatial velocity of 16 mm/s, which caused the capsule to deviate wildly from desired, which can be particularly observed in the \hat{x},\hat{z} plane.

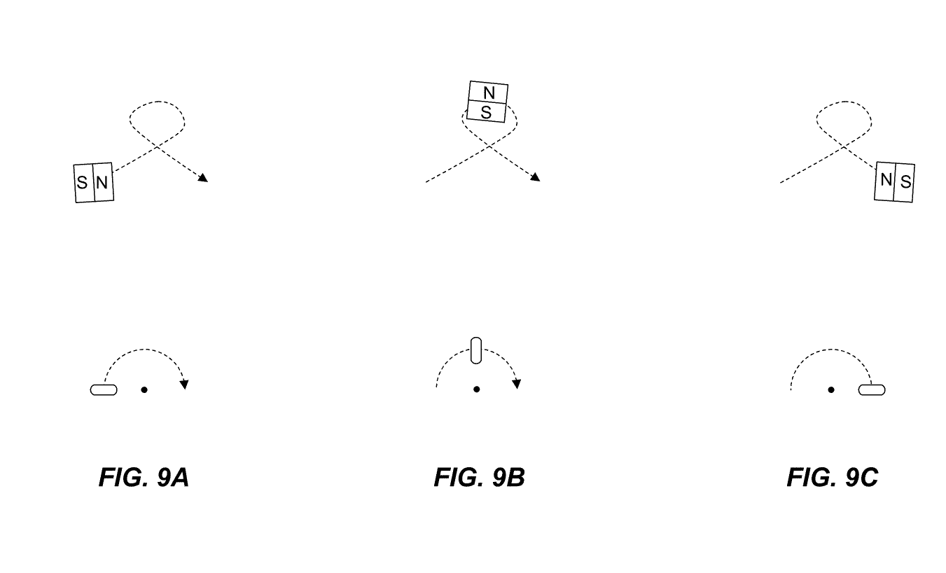

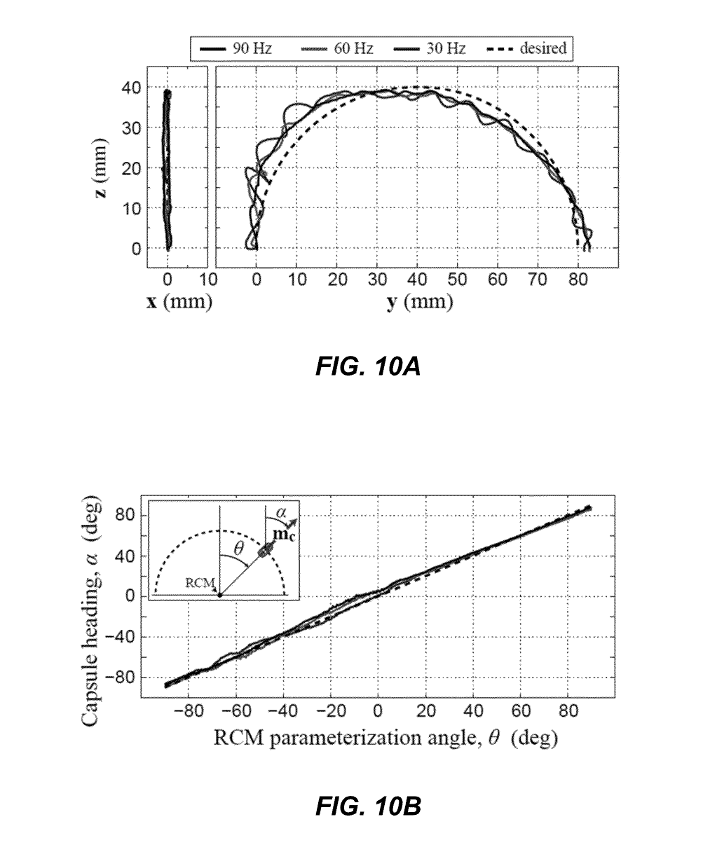

The vision system used to track the 3-DOF capsule position may not be feasible for clinical use. Existing clinically relevant localization strategies include RF triangulation, magnetic methods, and CT scan or x-ray fluoroscopy. In some experiments, the mockup capsule's position was localized at 90 Hz by the vision system. Clinically feasible localization methods may not provide the capsule's position at high rates (one known method can perform 3D position-tracking at approximately 50 Hz). The ability to actuate a mockup capsule with reduced 3D localization update frequencies is illustrated in FIGS. 9A-9C, which shows the mockup capsule performing a remote center-of-motion (RCM) maneuver, requiring the capsule's position to rotate around (while simultaneously pointing at) a fixed point in space. The update frequency of the tracking system (including the extended Kalman filter) was reduced to 60 Hz and 30 Hz, in order to simulate the update rate of a more clinically relevant localization method. FIGS. 9A-9C show images of the capsule performing the RCM maneuver with 90 Hz localization rate, while FIG. 10A shows the 3D position-tracking performance of the capsule for localization rates of 30, 60, and 90 Hz. The average position-tracking error for the localization rates of 30, 60, and 90 Hz was 2.7, 2.2, and 2.1 mm, respectively. The capsule's heading-tracking performance is shown in FIG. 10B. The heading-tracking performance tended to vary less with the localization frequency. The capsule moved at approximately 2 mm/s and completed the RCM maneuver in 62 s.

The mockup capsule was also transitioned from a configuration that forced the robot manipulator to enter its wrist singularity, to a configuration where the capsule pointed in the −\hat{z} direction by rotating the desired capsule direction 10° around the −ŷ axis, while simultaneously keeping the capsule's position in space stationary. In this example, the weight matrix W=diag([1, 1, 1, 20, 1, 1]) and the bound r=0.04 radians, which penalizes motion in the manipulator's fourth joint more than the others. In the initial robot manipulator configuration at time t=0 s, the desired change in the capsule direction would require the fourth joint of the robot manipulator to rapidly rotate π/2 radians. Rather than rapidly rotating the fourth joint, the constraint |Wδq|≦r penalized the fourth joint's velocity and forced the manipulator to first rotate the capsule about the \hat{z} axis before rotating about the ŷ axis as desired. This demonstrated the ability of the controller to balance desired changes in the capsule's configuration that may conflict with the robot manipulator's kinematics in a singularity.

In addition, the mockup capsule followed a U-shaped trajectory, with the desired capsule dipole moment pointing in a direction that forced the robot manipulator into its wrist singularity. The robot manipulator's joint configuration was nearly in the wrist-singular configuration throughout the trajectory. In this demonstration, the Jacobian J_{FA} was ill-conditioned and caused the inverse-kinematics approach using the psuedoinverse (15) to break down, which would result in incorrect magnetic forces being applied to the capsule. By solving the inverse kinematics using the formulation (20)-(22), control over the applied magnetic force was maintained but at the sacrifice of the capsule's heading.

It is to be understood that the above-referenced embodiments are illustrative of the application for the principles of the present invention. Numerous modifications and alternative arrangements can be devised without departing from the spirit and scope of the present invention while the present invention has been shown in the drawings and described above in connection with the exemplary embodiment(s) of the invention. It will be apparent to those of ordinary skill in the art that numerous modifications can be made without departing from the principles and concepts of the invention as set forth in the claims.

What is claimed is:

1. A method of manipulating an untethered magnetic device, comprising:
positioning a magnet actuator and an untethered magnetic device proximate one another such that the untethered magnetic device is within a constant magnetic field of the magnet actuator;
determining a position of the untethered magnetic device relative to the magnet actuator;
identifying at least one of a desired heading, position, and velocity of the untethered magnetic device;
calculating at least one of a magnetic field and a magnetic force to be applied to the untethered magnetic device to achieve the at least one of a desired heading, position, and velocity; and
moving the magnetic actuator to apply the calculated at least one of a magnetic field heading and a magnetic force to the untethered magnetic device via the magnetic field, wherein the magnetic field heading is operable to orient the untethered magnetic device in the desired device heading and the magnetic force is operable to translate the untethered magnetic device to the desired position and/or at the desired velocity.

2. The method of claim 1, wherein the desired heading and position of the untethered magnetic device are calculated independent of one another.

3. The method of claim 1, further comprising decoupling calculations of the magnetic field heading to determine the desired device heading and the magnetic force to determine the desired position and/or velocity.

4. The method of claim 1, wherein moving the actuator further comprises causing the untethered magnetic device to move in five degrees of freedom.

5. The method of claim 1, further comprising sacrificing the ability to control the untethered magnetic device's heading to maintain control over the untethered magnetic device's position when a robotic manipulator moving the magnetic actuator enters a kinematic singularity.

6. The method of claim 1, wherein the magnet actuator comprises a permanent magnet and a plurality of electromagnets, the method further comprising using the permanent magnet as a coarse control device for controlling the untethered magnetic device, and using the plurality of electromagnets in combination with the permanent magnet to provide fine corrections to the coarse control of the permanent magnet.

7. The method of claim 1, wherein estimating the position of the magnetic capsule endoscope includes an estimated solution based on an environment model.

8. The method of claim 1, wherein the untethered magnetic device is a magnetic capsule endoscope.

9. The method of claim 1, wherein at least one of the untethered magnetic device's desired heading, position, and velocity is provided by a human user.

10. The method of claim 1, wherein at least one of the untethered magnetic device's desired heading, position, and velocity is provided by a processing system.

11. A computer implemented method of controlling a magnet actuator to manipulate an untethered magnetic device, comprising:
under control of a processor and memory configured with executable instructions,
identifying a relative position of an untethered magnetic device and a magnet actuator, the untethered magnetic device being within a constant magnetic field of the magnet actuator;
identifying at least one of a desired heading, position, and velocity of the untethered magnetic device;
calculating at least one of a magnetic field heading and a magnetic force to be applied to the untethered magnetic device to achieve the at least one of a desired device heading, position, and velocity; and
transmitting instructions to move the magnet actuator to apply the calculated at least one of a magnetic field heading and a magnetic force to the untethered magnetic device via the magnetic field, wherein the magnetic field heading is operable to orient the untethered magnetic device in the desired heading and the magnetic force is operable to translate the untethered magnetic device to the desired position and/or at the desired velocity.

12. A system for manipulating an untethered magnetic device, comprising:
a magnet actuator configured to generate a constant magnetic field to influence at least one of a heading, a position, and a velocity of an untethered magnetic device;
a localization device to determine a position of the untethered magnetic device relative to the magnet actuator;
a control system to calculate at least one of a magnetic field heading and a magnetic force to be applied to the untethered magnetic device to achieve at least one of a desired heading, position, and velocity of the untethered magnetic device; and
a movement device configured to move the magnetic actuator to apply the calculated at least one of a magnetic field heading and a magnetic force to the untethered magnetic device via the magnetic field, wherein the magnetic field heading is operable to orient the untethered magnetic device in the desired heading and the magnetic force is operable to translate the untethered magnetic device to the desired position and/or at the desired velocity.

13. The system of claim 12, wherein the desired heading and position of the untethered magnetic device are calculated independent of one another.

14. The system of claim 12, further comprising decoupling calculations of the magnetic field heading to determine the desired device heading and the magnetic force to determine the desired position and/or velocity.

15. The system of claim 12, wherein the movement device comprises a robotic manipulator.

16. The system of claim 12, wherein the untethered magnetic device is a magnetic capsule endoscope.

17. The system of claim 12, wherein the localization device utilizes RF triangulation, magnetics, optics, CT scan, x-ray fluoroscopy, or combinations thereof.

18. The system of claim 12, wherein the magnet actuator comprises a single permanent magnet.

19. The system of claim 12, wherein the magnet actuator comprises an electromagnet configured to generate a constant magnetic field when energized.

20. The system of claim 12, wherein the magnet actuator includes a plurality of permanent magnets having a composite magnetic dipole.
